(19) United States
(12) Patent Application Publication
(10) Pub. No.: US 2012/0083516 A1
(43) Pub. Date: Apr. 5, 2012

(54) Nasal sinus spray for treatment of sinus headache and method of using same

(76) Inventor: Lyndon Mansfield, El Paso, TX (US)

(21) Appl. No.: 12/931,969

(22) Filed: Feb. 15, 2011

Related U.S. Application Data
(60) Provisional application No. 61/388,243, filed on Sep. 30, 2010.

(51) Int. Cl. A61K 31/4174; A61P 11/02; A61P 9/08; A61P 29/00; A61P 23/02

(52) U.S. Cl. 514/400

(57) ABSTRACT

Disclosed is a nasal spray containing the combination of a vasoconstrictor and a topical local anesthetic.

This application claims the benefit of the filing date, Feb. 16, 2010, of Provisional Application No. 61/388,243.

### BACKGROUND OF THE INVENTION

1. Field of the Invention

This invention relates to a nasal spray for the treatment of sinus headaches.

2. Description of the Prior Art

Sinuses are air filled cavities located in the forehead, cheekbones and behind the bridge of the nose. It is believed that the role of a sinus cavity is to make the skull lighter. Additionally, the sinus cavity assist in warming and humidifying the air inhaled before such air enters the lungs.

Sinuses have only a single portal for both entrance and exit of air and these are called the sinus antra. Sinus headaches are associated with a swelling of the sinus antra. Pain occurs in the affected region as a result of air, pus and mucus being trapped within the inflamed sinus cavities. Sinus headaches are also referred to as a vacuum or barometric headache. They are generally associated with dull, deep, throbbing pain in the front of the head and face. Bending down or leaning over generally makes the pain worse, as does cold and damp weather. This pain can be disabling and for this reason, immediate cessation is desired.

The main cause of sinus headaches is attributed to sinus infection. With a sinus infection, the mucosa, the membrane-like lining that covers the nasal passages and sinuses becomes inflamed. Normally the mucus produced by the sinuses is thin, colorless and has a slippery feel. But when there is inflamation, thick glue like sinus infection mucus starts forming. In this form, the mucus has difficulty in draining from the ostium, which is the orifice where mucus is pushed out of the sinus cavity by small hair like protrusions called cilia. The congestion of trapped, abnormally thick mucus results in pressure build up.

Treatment of sinus headaches is usually directed toward symptom relief and the treatment of any infection that may be causing the sinus cavities to become inflamed. In the case of acute sinus infection, doctors usually prescribe a course of antibiotics for about 10 days to get rid of the bacterial infection. Additionally, pain killing drugs such as acetaminophen or ibuprofen can be prescribed. There are many chronic sinus infection sufferers who get sinus headaches on a daily basis where this kind of treatment is not effective. Antibiotics are not effective because the swelling is not the result of bacterial infection. The mucosa of such people is constantly inflamed and as a result, the thick glue like mucus is being constantly produced and drainage is impaired leading to sinus pressure.

There are many types of treatment for sinus headaches disclosed in the prior art to include herbal or holistic approaches. Examples of herbal or holistic approaches included steam inhalation (which could include herbal additives) and nasal irrigation with a syringe. Medical treatments have historically included the use of antibiotics for the infection with possibly antihistamines, decongestants (such as Sudafed, Actifed or Afrin), pain relievers (such as aspirin, acetaminophen, ibuprofen or naproxen). Also disclosed in the prior art is the use of nasal costicosteroids, either in the form of spray or tablets, which reduces inflammation and includes such prescribed medications as Flonase, Nasonex, and Nasacort. Examples of over the counter medications for this same category of treatment include Benadryl, Chlor-Trimeton, Tavist, Claritin. What these prior medications do not disclose is the use of a topical local anesthetic in combination with a vasoconstrictor.

### SUMMARY OF THE INVENTION

It is, therefore, an object of the present invention to provide a sinus headache spray that effective reduces inflammation of the sinus cavities while simultaneously providing immediate relief to the sinus headache pain. It is also an object of this invention to provide for a method of use of the sinus headache spray. Other objects and advantages of the present invention will become apparent from the following detailed description which set forth certain embodiments of the invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

Vasoconstrictors work to narrow the blood vessels which in turn results in less blood flow. In terms of sinus headaches, a vasoconstrictor provides relief because it acts to reduce inflammation. While any vasoconstrictor can be used, oxymetazoline is utilized in the present invention.

The local anesthetic used in the present invention provides immediate relief because it acts to numb the tissue. While any local anesthetic can be used, lidocaine is used in the present invention. Specifically, a mixture of 5 ml of 2% lidocaine is combined with 15 ml of oxymetazoline.

1. A nasal spray comprising a vasoconstrictor and a topical local anesthetic.

2. A nasal spray as in claim 1 wherein the spray comprises a mixture of at least 5 ml of 2% lidocaine with 15 ml of oxymetazalone.

3. The method of using the spray of claim 1 comprising the steps of spraying said spray into the nose.
